(19) United States
(12) Patent Application Publication
(10) Pub. No.: US 2025/0367005 A1
(43) Pub. Date: Dec. 4, 2025

(54) SURGICAL INSTRUMENT

(71) Applicant: ENDOCON GMBH, Wiesenbach (DE)

(72) Inventor: Klaus NOTARBARTOLO, Wiesenbach (DE)

(21) Appl. No.: 18/875,787

(22) PCT Filed: Jun. 16, 2023
(86) PCT No.: PCT/EP2023/066236

§ 371 Date: Dec. 17, 2024

(30) Foreign Application Priority Data
Jun. 30, 2022 (DE) 10 2022 116 409.8

(51) Int. Cl. A61F 2/46

(52) U.S. Cl. CPC A61F 2/4603; A61F 2002/4681; A61F 2002/4692

(57) ABSTRACT

A surgical instrument for the revision of prostheses, comprising a handpiece in which a cylinder is arranged, wherein a piston element is associated with the cylinder, as well as a switching element and a chisel tool, wherein the chisel tool is arranged axially movably mounted at one end of the handpiece, wherein the chisel tool is fastened to the handpiece in a loss-proof manner, wherein the piston element is axially movably arranged in the cylinder, wherein the piston element can be set in motion by means of a fluid, wherein the piston element is configured to induce a pulse directed in the direction of the chisel tool for driving the chisel tool into a prosthesis stem interspace associated with the prosthesis, wherein the piston element is configured to induce a pulse, directed opposite to the chisel tool for driving the chisel tool out of the prosthesis stem interspace.

The invention relates to a surgical instrument for the revision of prostheses, comprising a handpiece in which a cylinder is arranged, wherein a piston element is associated with the cylinder, as well as a switching element and a chisel tool, wherein the chisel tool is arranged axially movably mounted at one end of the handpiece, wherein the chisel tool is fastened to the handpiece in a loss-proof manner, wherein the piston element is arranged axially movably in the cylinder, wherein the piston element can be set in motion by means of a fluid.

From EP 0 910 317 B1, a surgical instrument for the revision of prostheses is known, in which a piston element is pneumatically moved back and forth in a cylinder, wherein the piston element exerts an impact on a chisel tool axially mounted in the housing. Such a surgical instrument can be used to drive a chisel tool into an interspace between the prosthesis stem associated with the prosthesis and the recess made in a bone. The interspace is also referred to as the prosthesis stem interspace.

The chisel tool is usually shaped elongated and flexible. Such a design allows the chisel tool to penetrate very far into the prosthesis stem interspace. However, the disadvantage of this is that the chisel tool can get stuck in the prosthesis stem interspace. The chisel tool can then only be released with a considerable manual effort. For example, it is known to place a slotted hammer at the underside of the handpiece on the chisel tool and to loosen the chisel tool by controlled hammer blows from a second hammer. However, there is a risk of injury to the bones adjacent to the prosthesis and the procedure is time-consuming and not ergonomic for the surgeon.

The object of the invention is to provide a surgical instrument for the revision of prostheses which offers improved handleability.

This object is achieved using the features of claim 1. The dependent claims make reference to advantageous embodiments.

The surgical instrument according to the invention for the revision of prostheses comprises a handpiece in which a cylinder is arranged, wherein a piston element is associated with the cylinder, as well as a switching element and a chisel tool, wherein the chisel tool is arranged axially movably mounted at one end of the handpiece, wherein the chisel tool is fastened to the handpiece in a loss-proof manner, wherein the piston element is arranged axially movably in the cylinder, wherein the piston element can be set in motion by means of a fluid, wherein the piston element is configured to induce a pulse directed in the direction of the chisel tool for driving the chisel tool into a prosthesis stem interspace associated with the prosthesis, wherein the piston element is configured to induce a pulse directed opposite to the chisel tool for driving out the chisel tool from the prosthesis stem interspace.

By means of the pulse that can be generated in a controlled manner by means of a fluid and piston element both for driving the chisel tool into and driving it out of the prosthesis stem interspace associated with the prosthesis, a repeatability of the pulse is achievable. Gases and liquids are referred to as fluids. In particular, the piston element can be set in motion pneumatically or hydraulically. Possible fluids that can be used include compressed air or nitrogen, for example. In principle, it is conceivable that the fluid is a hydraulic fluid.

The pulse that can be generated to drive out the chisel tool can be used in case, for example, the chisel tool becomes jammed in the prosthesis stem interspace and is no longer freely movable. By means of pulses that can be generated in a controlled manner, also for driving out, the work process is standardized and simplified, process and work safety is increased and a consistent quality of the entire work process can be achieved. The time required for revision of prostheses is reduced, while at the same time the durability of the surgical instrument is increased by reducing possible misuse. Moreover, the patient's safety is increased, as damage to the prosthesis stem interspace and the surrounding body parts is reduced.

An advantageous embodiment of the invention provides for the piston element to be selectively configurable by means of the switching element for driving in or driving out the chisel tool. By means of this measure, the user is enabled to easily select the operating mode, which further increases the handleability of the surgical instrument.

A pulse transmitter can be associated with the cylinder, wherein the pulse transmitter is associated with the end face associated with the chisel tool, so that the pulse transmitter is arranged between the piston element and the chisel tool, wherein the pulse transmitter is configured to transmit a pulse from the piston element to the chisel tool, wherein the pulse transmitter seals the end face of the cylinder on the side facing the chisel tool. The pulse transmitter is typically a high-quality component and has good guiding properties in order to perform a precise movement relative to the cylinder. Furthermore, the pulse transmitter offers a receiving element for the chisel tool so that different chisel tools can be inserted without having to equip each chisel tool with high-quality guiding properties. This reduces the costs in the case of changing chisel tools while maintaining the quality standards.

The cylinder can be a tubular element that is inserted into the handpiece. The cylinder is preferably dimensioned in such a way that the piston element is arranged inside the cylinder so that it can move along the longitudinal axis of the cylinder. The gap between the piston element and the cylinder jacket is preferably designed in such a way that the piston element can be moved by a fluid.

A first and a second dynamic pressure chamber can be associated with the cylinder, wherein both dynamic pressure chambers can be connected to the cylinder in a flow-conducting manner, wherein a fluid source is associated with the cylinder. Depending on the direction of movement of the piston element, the first or second dynamic pressure chamber serves, among other things, to receive the fluid displaced by the piston element, wherein the displaced fluid is compressed in the respective dynamic pressure chamber. Preferably, the first and/or the second dynamic pressure chamber surround the cylinder at least partially along the longitudinal axis of the cylinder in a coaxial ring shape. The fluid source can be used to convey a fluid and serves as a drive means for the piston element. By means of this measure, hazardous liquids as a drive means can be avoided, for example, which can further increase process and work safety. The embodiment with dynamic pressure chambers is particularly suitable for compressible, gaseous fluids as the working medium. When using incompressible fluids, it is necessary for the dynamic pressure chambers to have a variable volume.

The cylinder can comprise a first channel on the side facing away from the chisel tool, wherein the cylinder is flow-connectable to the fluid source preferably through the first channel. On the side facing away from the chisel tool means, in the sense of the invention, that the channel can be arranged on the end face of the cylinder facing away from the chisel tool and/or in the jacket surface of the cylinder. A fluid can flow into the cylinder through this first channel in order to move the piston element in the direction of the chisel tool. This measure also serves to further increase process and work safety.

The cylinder can comprise a second channel on the side facing the chisel tool, wherein the cylinder is flow-connectable to the first dynamic pressure chamber preferably through the second channel. On the side facing the chisel tool means, in the sense of the invention, that the channel can be arranged on the end face of the cylinder facing the chisel tool and/or in the jacket surface of the cylinder. Through this second channel, a fluid exchange can take place between the cylinder and the first dynamic pressure chamber.

The first dynamic pressure chamber can comprise a third channel, wherein the first dynamic pressure chamber is flow-connectable to the fluid source preferably through the third channel. A fluid can flow from the fluid source into the first dynamic pressure chamber via the third channel. The third channel is preferably arranged on the side of the first dynamic pressure chamber facing away from the chisel tool. This allows shorter or more compact channel structures for the fluid to be achieved.

The cylinder can comprise a fourth channel on the side facing away from the chisel tool, wherein the cylinder is flow-connectable to the second dynamic pressure chamber preferably through the fourth channel. Through this fourth channel, a fluid exchange can take place between the cylinder and the second dynamic pressure chamber.

The first, third and fourth channels can be optionally closable. The first, third and fourth channels can be opened or closed independently of each other. Closable in the sense of the invention means that a channel is closed in a fluid-tight manner. At least this means a fluid tightness in such a manner, so that the intended sealing function can be achieved. Leakage can be tolerated if the function is not adversely affected.

For driving the chisel tool into the prosthesis stem interspace, the first and second channels can be in an open state and the third and fourth channels in a closed state. For driving in, the piston element exerts a pulse on the pulse transmitter, which pulse is transmitted to the chisel tool. The movement of the piston element to drive the chisel tool into the prosthesis stem interspace can be divided into two phases. During the first phase, the fluid is conveyed from the fluid source through the first channel into the cylinder and thus accelerates the piston element in the direction of the side facing the chisel tool until pulse transmission to the pulse transmitter takes place. Thereby, the fluid in the cylinder between the piston element and the side facing the chisel tool is conveyed by the moving piston element through the second channel into the first dynamic pressure chamber and compressed. During the second phase, conveying of fluid from the fluid source is interrupted. Furthermore, the fluid compressed in the first dynamic pressure chamber expands and flows back into the cylinder through the second channel and drives the piston element to the side of the cylinder facing away from the chisel tool in accordance with the initial position of the movement for driving in the chisel tool.

For driving out the chisel tool from the prosthesis stem interspace, the first channel can be in a closed state and the second, third and fourth channels can be in an open state. For driving out, the piston element exerts a pulse in the opposite direction to the chisel tool. The movement of the piston element to drive out the chisel tool from the prosthesis stem interspace can also be divided into two phases. During the first phase, the fluid is conveyed from the fluid source through the third channel into the first dynamic pressure chamber and further through the second channel into the cylinder. The fluid accelerates the piston element in the direction of the side facing away from the chisel tool until pulse transmission to the cylinder takes place. The fluid in the cylinder between the piston element and the side facing away from the chisel tool is conveyed by the moving piston element through the fourth channel into the second dynamic pressure chamber and compressed there. Preferably, the fourth channel is arranged in such a way that no fluid cushion, in particular no gas pressure cushion, can build up between the piston element and the side of the cylinder facing away from the chisel tool. This can further improve the pulse transmission from the piston element to the cylinder. During the second phase, conveying of fluid from the fluid source is interrupted. The fluid compressed in the second dynamic pressure chamber expands and flows back into the cylinder through the fourth channel and drives the piston element to the side of the cylinder facing the chisel tool in accordance with the initial position of the movement for driving out the chisel tool.

The fluid source can generate pulsed pressure surges, wherein the repetition frequency is preferably in the range between 1 and 40 Hz. It is particularly preferred if the repetition frequency is in the range between 2 and 20 Hz. These pulsed pressure surges enable targeted repetitions of the pulse in a small time window. Compared to manual release efforts of the surgical instrument, higher repetition frequencies are possible. By means of these measures, the process and work safety and the handleability are further improved.

The switching element can be designed as a rotary handle, wherein the switching element has a channel structure which establishes a flow connection either between the fluid source and the cylinder by means of the first channel or between the fluid source and the first dynamic pressure chamber by means of the third channel. The rotary handle improves handleability, as the operator can choose between driving in and driving out by means of a rotary handle. Advantageously, only small turns are required to select the switch position of the rotary handle. Elaborate grip changing or re-gripping of the surgical instrument or changing the viewing angle is not necessary. Furthermore, engaging means can be provided to assign defined positions of the rotary handle to the individual switch positions.

The chisel tool can be arranged on the handpiece in a tool-free interchangeable manner. The chisel tool is preferably fastened using a bayonet catch. In addition to the tool-free interchangeability, the short processing time for establishing and releasing the mechanical connection is also advantageous.

A magnet holder can be arranged on the side facing away from the chisel tool, wherein the piston element comprises a ferromagnetic material. The magnet holder is designed in such a way that it can hold the piston element in its end position facing away from the chisel tool. This counteracts any unwanted movement of the piston element in the cylinder. By supplying fluid from the fluid source, the holding force of the magnetic holder can be overcome and the piston element can be moved. By means of this measure, the initial position of the piston element is fixed, so that increased process reliability can be achieved.

An advantageous embodiment of the invention provides that the surgical instrument comprises a display element. The display element is preferably digital. The information that can be displayed preferably comprises the fluid pressure applied, the set repetition rate and possible error messages. By means of this measure, the user receives feedback on settings made or other useful information, and therefore this measure serves to further increase handleability.

One embodiment of the surgical instrument according to the invention is explained in more detail with reference to the figures. These show, in each case schematically:

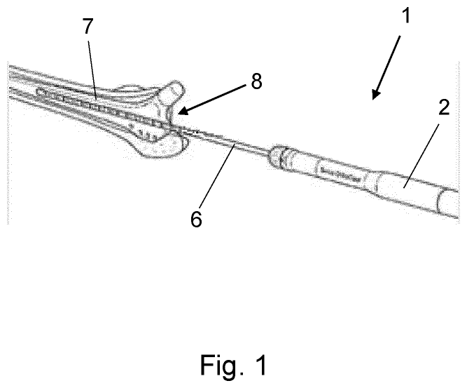

FIG. 1 a surgical instrument for the revision of prostheses;

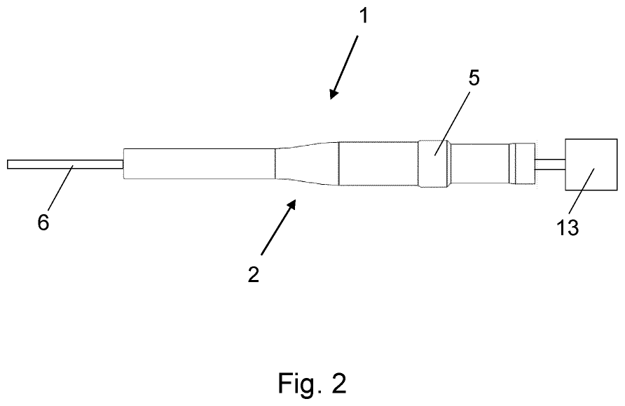

FIG. 2 the surgical instrument with fluid source;

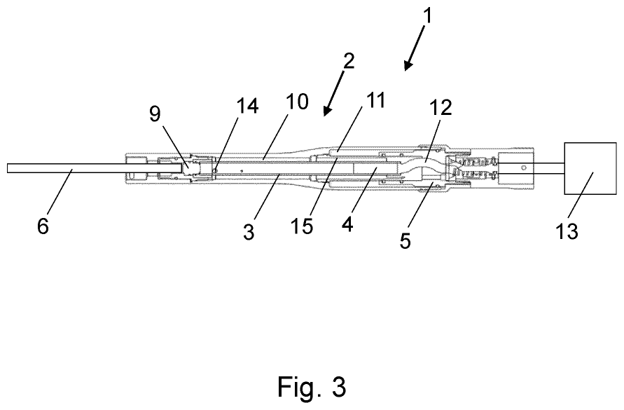

FIG. 3 a sectional view of the surgical instrument in a state for driving in the chisel tool;

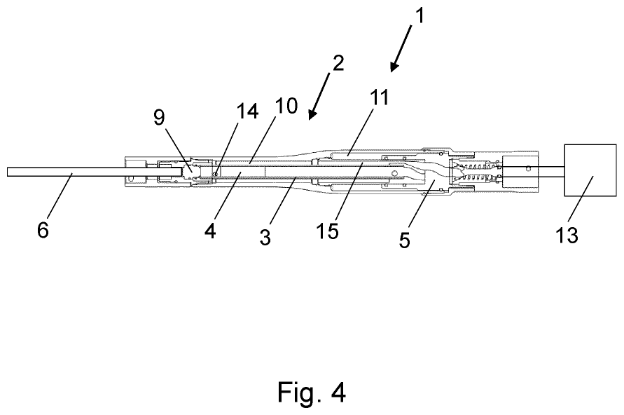

FIG. 4 a first sectional view of the surgical instrument in a state for driving out the chisel tool;

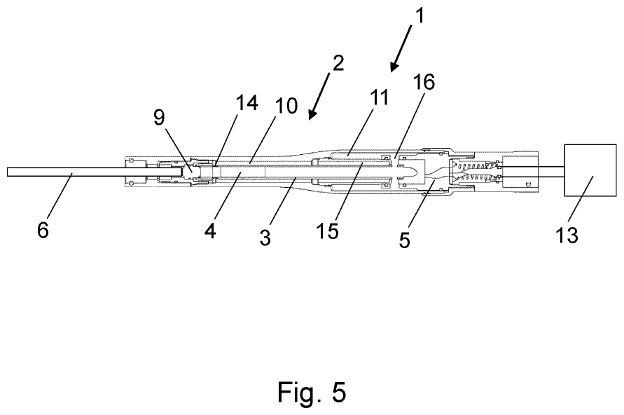

FIG. 5 a second sectional view of the surgical instrument in a state for driving out the chisel tool;

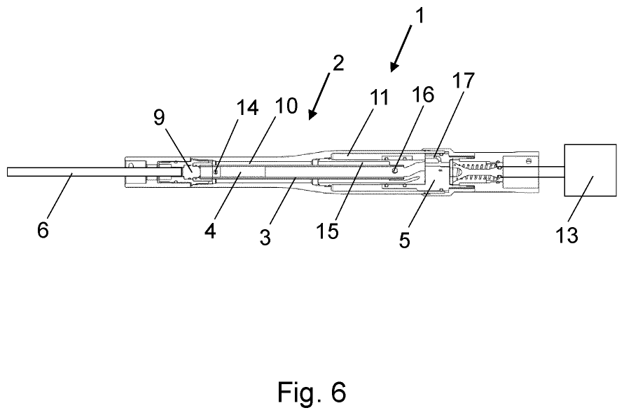

FIG. 6 a third sectional view of the surgical instrument in a state for driving out the chisel tool.

FIG. 1 shows a surgical instrument 1 for the revision of prostheses, in particular of hip joint endoprostheses. The surgical instrument 1 comprises a handpiece 2 and a chisel tool 6. The chisel tool 6 is axially movably mounted at one end of the handpiece 2 and fastened to the handpiece 2 in a loss-proof manner. The chisel tool 6 is fastened by means of a bayonet catch and can be exchanged without tools.

For revision, the chisel tool is driven into the prosthesis stem interspace 8 associated with the prosthesis 7. This loosens the prosthesis 7 fastened to the bone. However, the surgical instrument 1 according to the invention also enables the chisel tool 6 to be driven out of the prosthesis stem interspace 8 associated with the prosthesis 7. This is particularly advantageous in case the chisel tool 6 has penetrated too deeply into the prosthesis stem interspace 8 and gets stuck there. FIG. 1 shows the revision procedure, wherein the chisel tool 6 is partially driven into the prosthesis stem interspace 8.

FIG. 2 shows the surgical instrument 1 according to FIG. 1 for the revision of prostheses with a fluid source 13. In the present case, the fluid source 13 is a compressed air source. The compressed air can be supplied by a compressor. Alternatively, it is also conceivable to take a compressed gaseous medium such as nitrogen from a pressure accumulator.

The surgical instrument 1 comprises a handpiece 2, a switching element 5 and a chisel tool 6. Via the switching element 5, the setting can be selectively set such that the surgical instrument 1 can be used for driving in the chisel tool 6 or driving it out of the prosthesis stem interspace 8. The drive means for generating the pulse is a gaseous fluid in the form of compressed air, which is provided via the fluid source 13. The fluid source 13 generates pulsed compressed air bursts, wherein the repetition frequency is in the range between 1 and 40 Hz. The compressed air is transmitted from the fluid source 13 to the handpiece 2 via a compressed air hose.

FIG. 3 shows a sectional view of the surgical instrument 1 according to FIG. 2 in a state for driving in the chisel tool 6. The surgical instrument 1 comprises a handpiece 2, a switching element 5 and a chisel tool 6.

A cylinder 3 is arranged in the handpiece 2, wherein the cylinder 3 is a tubular element which is inserted into the handpiece 2. A piston element 4 is associated with the cylinder 3, wherein the piston element 4 is arranged in the cylinder 3 in an axially movable manner and can be set in motion by means of compressed air. The piston element 4 is configured to induce a pulse directed in the direction of the chisel tool 6 to drive the chisel tool 6 into the prosthesis stem interspace 8 associated with the prosthesis 7. Furthermore, the piston element 4 is configured to induce a pulse in the opposite direction to the chisel tool 6 to drive out the chisel tool 6 from the prosthesis stem interspace 8.

A pulse transmitter 9 is associated with the cylinder 3. The pulse transmitter 9 is associated with the end face associated with the chisel tool 6 and is arranged between the piston element 4 and the chisel tool 6. The pulse transmitter 9 is configured to transmit a pulse from the piston element 4 to the chisel tool 6, wherein the pulse transmitter 9 seals the end face of the cylinder 3 on the side facing the chisel tool 6.

The cylinder 3 comprises a first channel 12 on the side facing away from the chisel tool 6. The cylinder 3 is flow-connected to the fluid source 13 through the first channel 12.

A first dynamic pressure chamber 10 and a second dynamic pressure chamber 11 are associated with cylinder 3. Both dynamic pressure chambers 10, 11 can be connected to the cylinder 3 in a flow-conducting manner. The cylinder 3 comprises a second channel 14 on the side facing the chisel tool 6 and is flow-connected to the first dynamic pressure chamber 10 through the second channel 14.

The first dynamic pressure chamber 10 comprises a third channel 15 and is flow-connectable to the fluid source 13 through the third channel 15. In the depicted state, the third channel 15 is closed and the first dynamic pressure chamber 10 is not fluid-connected to the fluid source 13.

Via the switching element 5, the piston element 4 is selectively configurable to drive in or drive out the chisel tool 6. For this purpose, the switching element 5 is designed as a rotary handle and has a channel structure. Through this channel structure, a flow connection can be selectively set or interrupted between the fluid source 13 and the cylinder 3 by means of the first channel 12, between the fluid source 13 and the first dynamic pressure chamber 10 by means of the third channel 15 and between the cylinder 3 and the second dynamic pressure chamber 11 by means of the fourth channel. The first channel 12, the third channel 15 and the fourth channel 16 (not depicted here) are therefore selectively closable.

The first channel 12 is open so that there is a flow connection between the fluid source 13 and the cylinder 3 by means of the first channel 12. The third channel 15 is closed. The fourth channel 16 is also closed and is not depicted in this figure due to the complex geometry of the channel structure of the rotary handle.

For driving in the chisel tool 6, the first channel 12 and the second channel 14 are in an open state. The third channel 15 and the fourth channel are in a closed state. For driving in, the piston element 4 is accelerated by the compressed air burst in the direction of the pulse transmitter 9 and finally exerts a pulse on the pulse transmitter 9, which pulse in turn is transmitted to the chisel tool 6. To move the piston element 4 for driving in the chisel tool 6, compressed air is conveyed from the fluid source 13 through the first channel 12 into the cylinder 3 and thus accelerates the piston element 4 in the direction of the side facing the chisel tool 6 until pulse transmission to the pulse transmitter 9 takes place. At the same time, the compressed air in the cylinder 3 is conveyed between the piston element 4 and the side facing the chisel tool 6 by the moving piston element 4 through the second channel 14 into the first dynamic pressure chamber 10 and compressed. As soon as the compressed air burst emanating from the fluid source 13 ends, the compressed air compressed in the first dynamic pressure chamber 10 expands and flows through the second channel 14 back into the cylinder 3 and drives the piston element 4 back to the side of the cylinder 3 facing away from the chisel tool 6, corresponding to the initial position of the movement for driving in the chisel tool 6.

FIGS. 4, 5 and 6 show sectional views of the surgical instrument 1 depicted in Figure 3 in a state for driving out the chisel tool 6.

For driving out the chisel tool 6, the first channel 12 is in a closed state and the second channel 14, the third channel 15 and the fourth channel 16 are in an open state. Due to the complex geometry of the channel structure of the rotary handle, the first channel 12 is not depicted in these figures.

The third channel 15 is open so that there is a flow connection between the fluid source 13 and the dynamic pressure chamber 10 via the third channel 15.

For driving out, the piston element 4 exerts a pulse opposite to the chisel tool 6. To move the piston element 4 for driving out the chisel tool, a compressed air burst is conveyed from the fluid source 13 through the third channel 15 into the first dynamic pressure chamber 10 and further through the second channel 14 into the cylinder 3. The compressed air accelerates the piston element 4 in the direction of the side facing away from the chisel tool 6 until pulse transmission to the cylinder 3 takes place. The compressed air in the cylinder 3 between the piston element 4 and the side facing away from the chisel tool 6 is conveyed by the moving piston element 4 through the fourth channel 16 into the second dynamic pressure chamber 11 and compressed there.

The compressed air, which is located between the opening to the fourth channel 16 in the cylinder 3 and the side of the cylinder 3 facing away from the chisel tool 6, is conveyed through the fifth channel 17 into the dynamic pressure chamber 11 when the piston element 4 closes the opening of the fourth channel 16. The fifth channel 17 is open when the piston element 4 is driven out, so that there is a flow connection between the cylinder 3 and the dynamic pressure chamber 11 by means of the fifth channel 17. Due to the complex geometry, the fifth channel 17 is only visible in FIG. 6. The fifth channel 17 advantageously prevents an air cushion from building up between the piston element 4 and the side of the cylinder 3 facing away from the chisel tool 6 when the piston element 4 is driven out. By means of this measure, the pulse transmission can be improved. Subsequently, the compressed air compressed in the second dynamic pressure chamber 11 expands and flows back into the cylinder 3 through the fourth channel 16 and drives the piston element 4 to the side of the cylinder 3 facing the chisel tool 6 corresponding to the initial position of the movement to drive out the chisel tool 6.

FIG. 5 shows a second sectional view of the surgical instrument 1 depicted in FIG. 3 and FIG. 4. The surgical instrument 1 is in a state for driving out the chisel tool 6 in accordance with FIG. 4. In FIG. 5, the sectional view is in a different plane.

FIG. 6 shows a third sectional view of the surgical instrument 1 depicted in FIG. 3, FIG. 4 and FIG. 5 with the sectional plane rotated by 90° compared to FIG. 5. The surgical instrument 1 is in a state for driving out the chisel tool 6 in accordance with FIG. 4 and FIG. 5.

In the views of FIGS. 5 and 6, the flow connection between the cylinder 3 and the second dynamic pressure chamber 11 through the fourth channel 16 is depicted, respectively.

What is claimed is:

1. A surgical instrument for the revision of prostheses, comprising a handpiece in which a cylinder is arranged, wherein a piston element is associated with the cylinder, as well as a switching element and a chisel tool, wherein the chisel tool is arranged axially movably mounted at one end of the handpiece, wherein the chisel tool is fastened to the handpiece in a loss-proof manner, wherein the piston element is axially movably arranged in the cylinder, wherein the piston element can be set in motion by means of a fluid, wherein the piston element is configured to induce a pulse directed in the direction of the chisel tool for driving the chisel tool into a prosthesis stem interspace associated with the prosthesis, characterized in that the piston element is configured to induce a pulse directed opposite to the chisel tool for driving the chisel tool from the prosthesis stem interspace.

2. The surgical instrument according to claim 1, wherein the piston element is selectively configurable by means of the switching element for driving in or driving out the chisel tool.

3. The surgical instrument according to claim 1, wherein a pulse transmitter is associated with the cylinder, wherein the pulse transmitter is associated with the end face associated with the chisel tool, so that the pulse transmitter is arranged between the piston element and the chisel tool, wherein the pulse transmitter is configured to transmit a pulse from the piston element to the chisel tool, wherein the pulse transmitter seals the end face of the cylinder on the side facing the chisel tool.

4. The surgical instrument according to claim 1, wherein the cylinder is a tubular element which is inserted into the handpiece.

5. The surgical instrument according to claim 1, wherein a first dynamic pressure chamber and a second dynamic pressure chamber are associated with the cylinder, wherein the dynamic pressure chambers can be connected to the cylinder in a flow-conducting manner, wherein a fluid source is associated with the cylinder.

6. The surgical instrument according to claim 5, wherein the cylinder comprises a first channel on the side facing away from the chisel tool, wherein the cylinder is flow-connectable to the fluid source through the first channel.

7. The surgical instrument according to claim 5, wherein the cylinder comprises a second channel on the side facing the chisel tool, wherein the cylinder is flow-connectable to the first dynamic pressure chamber through the second channel.

8. The surgical instrument according to claim 5, wherein the first dynamic pressure chamber comprises a third channel, wherein the first dynamic pressure chamber is flow-connectable to the fluid source through the third channel.

9. The surgical instrument according to claim 5, wherein the cylinder comprises a fourth channel on the side facing away from the chisel tool, wherein the cylinder is flow-connectable to the second dynamic pressure chamber through the fourth channel.

10. The surgical instrument according to claim 9, wherein the first, third and fourth channels are selectively closable.

11. The surgical instrument according to claim 10, wherein, for driving the chisel tool into the prosthesis stem interspace, the first and second channels are in an open state and the third and fourth channels are in a closed state.

12. The surgical instrument according to claim 11, wherein, for driving out the chisel tool from the prosthesis stem interspace, the first channel is in a closed state and the second, third and fourth channels are in an open state.

13. The surgical instrument according to claim 5, wherein the fluid source (generates pulsed compressed air bursts, wherein the repetition frequency is in the range between 1 and 40 Hz.

14. The surgical instrument according to claim 1, wherein the switching element is designed as a rotary handle, wherein the switching element has a channel structure, which, in a first position, establishes a flow connection between the fluid source and the cylinder by means of the first channel and interrupts a flow connection between the fluid source and the first dynamic pressure chamber by means of the third channel and interrupts a flow connection between the cylinder and the second dynamic pressure chamber by means of the fourth channel, or, in a second position, interrupts a flow connection between the fluid source and the cylinder by means of the first channel and establishes a flow connection between the fluid source and the first dynamic pressure chamber by means of the third channel and establishes a flow connection between the cylinder and the second dynamic pressure chamber by means of the fourth channel.
